(19) United States
(12) Patent Application Publication
(10) Pub. No.: US 2005/0193945 A1
(43) Pub. Date: Sep. 8, 2005

(54) Quiet automated spray tanning booth

(76) Inventors: Jerry Coffield, Brookville, OH (US); Kent McGuire, Brookville, OH (US)

Correspondence Address: Donald W. Meeker;Patent Agent, 924 East Ocean Front #E, Newport Beach, CA, 92661, US

(21) Appl. No.: 10/793,569

(22) Filed: Mar. 4, 2004

(51) Int. Cl. B05C 11/00; A61M 35/00

(52) U.S. Cl. 118/704; 118/696; 604/290

(57) ABSTRACT

An electric motor pressurized line and tank provide a cosmetic solution through a hose loop to an array of spaced stationary spray nozzles in a spray booth. A microprocessor automatically opens solenoid valves in the hose loop for spraying the cosmetic solution into the spray booth and drawing unused cosmetic solution back into the tank. A water spray and nozzle cleans the spray booth and a drainage tank with a pump forces drainage out a drain opening in the bottom of the booth and into the drainage tank.

### BACKGROUND OF THE INVENTION

1. Field of the Invention

The present invention relates to the field of automated spray booths for coating a human body and particularly to a spray booth for a cosmetic solution to simulate tanning using an electric pump to pressurize a tank containing the solution and, upon a user pressing a start button, using a microprocessor to control a solenoid valve to release the pressurized solution through stationary nozzles for providing a quieter automated spray booth with two controlled time periods of spray for a front and a back of a user and a programmed automatic water cleanout spray and drainage of the booth after use.

2. Description of the Prior Art

A tanned complexion is considered to be an attractive feature. And, a tan is still presently achieved primarily by exposing the skin to ultraviolet (hereinafter referred to as “UV”) radiation. However, the concern about damage to the skin, particularly skin cancer, caused by exposing the skin to UV radiation from the sun or other sources such as, for example, sun lamps, has caused an increased interest in products that can produce a “self-tan” without the use of UV radiation. As a result, compositions useful for inducing a tan without exposure to the sun have been introduced for many years. For example, U.S. Pat. No. 2,949,403 discloses that the compound dihydroxyacetone (hereinafter referred to as “DHA”) can be used for this purpose; since that report, DHA has been widely employed in commercial self-tanning products.

Prior art shows booths with atomizing systems for delivering DHA solutions, which uniformly coat the entire body or selected parts of the body of the person being coated. Many of them employ noisy air compressors and movable spray nozzles with many moving parts subject to breaking.

U.S. Patent Application #20030127542, published Jul. 10, 2003 by Cooper, claims a uniform metering system for spray applications. The metering system includes a pumping device having one or more cylinders, each cylinder having a piston therein that is moved by an automated drive system to produce uniform or proportional flow to a single spray nozzle or a plurality of nozzles. The cylinders are mounted between a common base and a common metering plate. Movement of the metering plate relative to the base causes the pistons to slide within the cylinders to provide pulse-free fluid pumping. A rinsing cylinder or dual-action cylinder can also be included to provide delivery of a rinsing agent or multiple liquids to one or more of the spray nozzles simultaneously or in sequence.

U.S. Pat. No. 5,922,333, issued Jul. 13, 1999 to Laughlin, provides a system for automatically coating the human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions.

U.S. Pat. No. 6,199,557, issued Mar. 13, 2001 to Laughlin, shows the method of and apparatus for automatically coating the human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions.

U.S. Pat. No. 6,251,374, issued Jun. 26, 2001 to Laughlin, claims the method, apparatus, and composition for automatically coating the human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions. An improved self-tanning composition is useful in conjunction with the system is also disclosed.

U.S. Pat. No. 6,298,862, issued Oct. 9, 2001 to Laughlin, describes the method of and apparatus for automatically coating the human body, which has fogging technology. In the system for coating human skin, a chemical composition, such as a cosmetic or medical formulation, is uniformly coated over the entire body or selected parts of the body of the person being coated. The system-includes atomization of the coating composition, containment of the atomized spray, and residual recovery, which together yield a novel method for applying chemical compositions.

U.S. Pat. No. 6,302,122, issued Oct. 16, 2001 to Parker, discloses an apparatus for automatic application of compositions to the skin. An apparatus is described for the coating of a human body with a tanning composition, such as a sunless tanning composition, wherein an arm with a plurality of nozzles thereon traverses within a booth to spray coat a body in the booth. The spray nozzles are oriented to avoid opposing air flows and excessive air flows which cause inefficient and uneven, dripping or streaking in the deposition of tanning composition on the skin. In another aspect, the apparatus provides for a foot rinser to rinse the feet of a user while the apparatus applies a sunless tanning composition to the user to avoid overly darkening the user's feet. In yet another aspect, the apparatus provides for a sanitation system to wash-down the spray booth between successive uses.

U.S. Pat. No. 6,305,384, issued Oct. 23, 2001 to Laughlin, indicates the system for automatically coating the human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions.

U.S. Pat. No. 6,554,208, issued Apr. 29, 2003 to Venuto, Sr., puts forth a tanning booth that has automated spray, which comprises a spray chamber having a spray system and a ventilation system. The spray system includes a compressor and stationary spray jets that are positioned 360 . degree. about a user, from head to toe. The spray system operates in a spray mode and a drying mode. In the spray mode, the composition is sprayed onto the user through the spray jets in the form of a mist. In the drying mode, air is blown through the jets to dry the user. The spray chamber also has a shower spray. In the rinse mode, chlorinated water is emitted from the shower spray to disinfect the spray chamber. The ventilation system includes exhaust fans that operate in the drying mode to draw air and remnant spray mist into a ventilation housing. The spray is filtered to create droplets that are siphoned by a sump pump as waste to a sewer system.

U.S. Pat. No. 6,416,747, issued Jul. 9, 2002 to Laughlin, concerns the method, apparatus, and composition for automatically coating the human body with plural components. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions. An improved self-tanning composition is useful in conjunction with system is disclosed. Also disclosed is a plural component self-tanning system.

U.S. Pat. No. 6,446,635, issued Sep. 10, 2002 to Laughlin, illustrates an automated system for coating human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions.

U.S. Pat. No. 6,443,164, issued Sep. 3, 2002 to Parker, is for an apparatus for the coating of a human body with a tanning composition, such as a sunless tanning composition, wherein an arm with a plurality of nozzles thereon traverses within a booth to spray coat a body in the booth. The spray nozzles are oriented to avoid opposing air flows and excessive air flows which cause inefficient and uneven, dripping or streaking in the deposition of tanning composition on the skin. In another aspect, the apparatus provides for a foot rinser to rinse the feet of a user while the apparatus applies a sunless tanning composition to the user to avoid overly darkening the user's feet. In yet another aspect, the apparatus provides for a sanitation system to wash-down the spray booth between successive uses.

U.S. Pat. No. 6,431,180, issued Aug. 13, 2002 to Laughlin, provides an automated system for coating the human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions.

U.S. Pat. No. 6,474,343, issued Nov. 5, 2002 to Laughlin, shows the method of and apparatus for automatically coating the human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions.

U.S. Pat. No. 6,468,508, issued Oct. 22, 2002 to Laughlin, claims the method, apparatus, and composition for automatically coating the human body and skin preconditioning system for use therewith. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions. An improved self-tanning composition is useful in conjunction with system is also disclosed. Also disclosed is a method of preconditioning the skin prior to applying the self-tanning composition.

U.S. Pat. No. 6,439,243, issued Aug. 27, 2002 to Laughlin, describes the method of and apparatus for automatically coating the human body. In the system for coating human skin, a chemical composition, such as a cosmetic or medical formulation, is uniformly coated over the entire body or selected parts of the body of the person being coated. The system includes atomization of the coating composition, containment of the atomized spray, and residual recovery, which together yield a novel method for applying chemical compositions.

U.S. Pat. No. 6,387,081, issued May 14, 2002 to Cooper, discloses a misting apparatus and method for electrostatically applying coating materials to skin surface. In one embodiment, the invention includes an enclosure; a mount positioned on the enclosure; an electrostatic nozzle connected to the mount, the electrostatic nozzle for passing the coating composition; and a grounding connection positioned inside the enclosure, the grounding connection capable of electrically grounding the human; wherein the coating composition passed through the electrostatic nozzle is depositable upon the human. Furthermore, in another embodiment, the enclosure includes a first wall wherein the mount is positioned on the first wall; and a second wall positioned substantially opposite the first wall, the second wall including a portion curved about an axis.

U.S. Pat. No. 5,460,192, issued Oct. 24, 1995 to McClain, indicates an apparatus for applying suntan lotion, which includes a liquid tight enclosure of generally cylindrical shape. The upper wall portions are tilted inwardly to form a central neck enclosure. A door in side walls of the enclosure is spring biased to the closed position. Suntan lotion is pumped through vertically spaced nozzles from a pump and supply source exterior of the enclosure. The pump is controlled by a revenue token device outside the apparatus with a time delay arrangement allowing the user to deposit tokens, enter the enclosure, and close the door before the lotion starts spraying for a predetermined time.

U.S. Pat. No. 5,664,593, issued Sep. 9, 1997 to McClain, puts forth an apparatus for applying suntan lotion, which includes a liquid tight enclosure of generally cylindrical shape. The upper wall portions are tilted inwardly to form a central neck enclosure. A door in side walls of the enclosure is spring biased to the closed position. Lotion mist ports are disposed about the interior of the enclosure and supplied from a lotion distribution tube, which in turn is supplied from at least one atomizing nozzle. A pump pumps the liquid lotion from a supply dispenser to the atomizing nozzles. As the lotion is pumped through the atomizing nozzle, it is formed into mist droplets which are communicated to the distribution pipe and then to the mist outlets in the chamber. The droplets contact the body of the person in the chamber and are applied to the skin in that manner. A recirculating pump is provided for evacuating the misted droplets and to recirculate them into the distribution tube by means of an impeller fan. Lotion that may condense inside the impeller is stored in a used lotion recovery reservoir. The pump and motor which drives the pump and empeller fan is operated by token operator control located outside the enclosure.

What is needed is a spray booth for a cosmetic solution to simulate tanning using an electric pump to pressurize a tank containing the solution and, upon a user pressing a start button, using a microprocessor to control a solenoid valve to release the pressurized solution through stationary nozzles for providing a quieter automated spray booth.

### SUMMARY OF THE INVENTION

An object of the present invention is to provide a spray booth for a cosmetic solution to simulate tanning using an electric pump to pressurize a tank containing the solution and, upon a user pressing a start button, using a microprocessor to control a solenoid valve to release the pressurized solution through stationary nozzles for providing a quieter automated spray booth with two controlled time periods of spray for a front and a back of a user.

Another object of the present invention is to provide a programmed automatic water cleanout spray of the booth and automatic pumping out of the waste water and solution from the bottom of the booth after use to prevent build-up of the solution on the surfaces of the booth and insure a clean booth for the next user.

One more object of the present invention is to provide a vertical series of stationary nozzles to cover the user from head to toe thereby eliminating the need for moving nozzles which can malfunction and break because of the moving parts.

In brief, the cosmetic tanning solution spray system of the present invention is unique in that it utilizes airless spray technology. The entire process is controlled by a microprocessor control system. The cosmetic solution is drawn from the 5 gallon cosmetic solution reservoir by a 100 PSI Shurflow model 100 diaphragm electric pump. After passing through a 0 to 100 PSI pressure gauge and flow check valve it is sent to the cosmetic solution spray control solenoid valve. An inline 2 gallon pressure storage tank insures that there will be adequate solution available when the microcontroller signals the start of the process. When the start button is pressed by the user, the processor enters a wait state, and then activates the cosmetic solution spray control solenoid valve and the solution flows to the cosmetic solution spray manifold. The solution is atomized through one or more T-Jet Cone Pattern SIS 0.1 or SIS 0.2 nozzles, preferably four stationary nozzles aligned vertically to cover the entire body from head to foot. Following the spray, the processor signals the solution relief solenoid to open allowing a small amount of the solution to return to the 5 gallon cosmetic solution reservoir; this also provides an opportunity for the user to turn around for even coverage. The solution spray/relief cycle starts again. A similar pump system pumps water to clean the booth after use and another similar pump system pumps out the waste water and solution from the bottom of the booth to a waste container for disposal.

An advantage of the present invention is that it provides a quiet cosmetic tanning spray booth.

Another advantage of the present invention is that it has a pre-programmed controlled amount of spray to adequately coat the user without wasting solution.

An additional advantage of the present invention is that it automatically cleans out the booth after each use.

One more advantage of the present invention is that it has no moving nozzle parts that can malfunction and break.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a side elevational diagrammatic view of the spray booth showing the vertical array of stationary nozzles for spraying the cosmetic solution on a user from head to toe;

FIG. 2 is a side elevational diagrammatic view of the spray booth combined with a schematic view of the operational components feeding the spray booth including the electric motors for pressurizing the tanks and the microprocessor for controlling the operation and timing of the solenoid valves to release the cosmetic solution to the vertical array of stationary spray nozzles, release the water to at least one top spray valve to clean the booth after use, and pump out the waste water and solution from the booth.

### BEST MODE FOR CARRYING OUT THE INVENTION

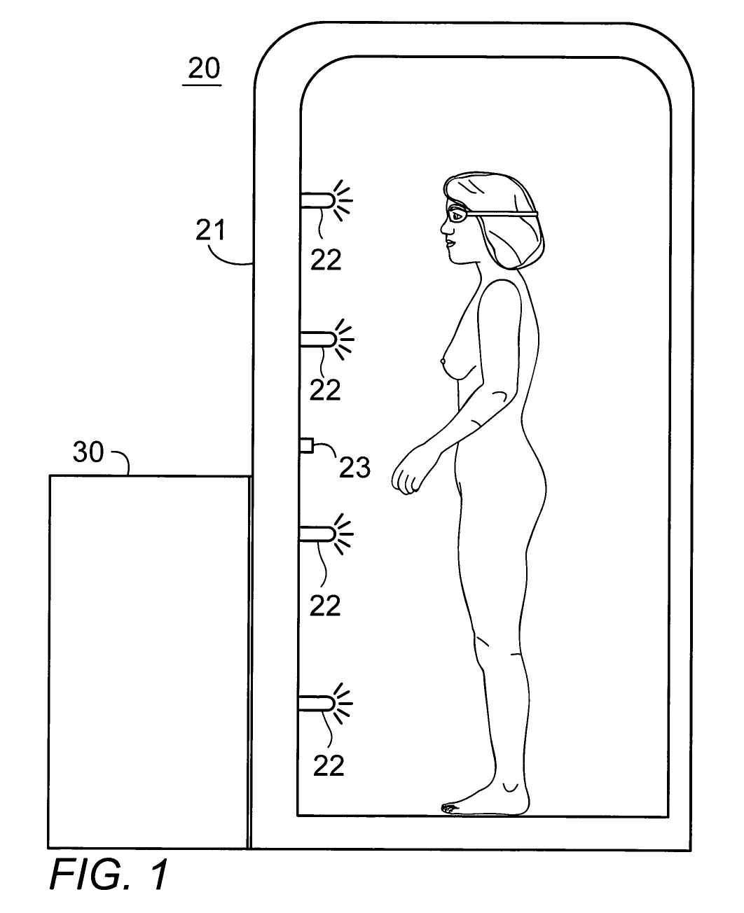

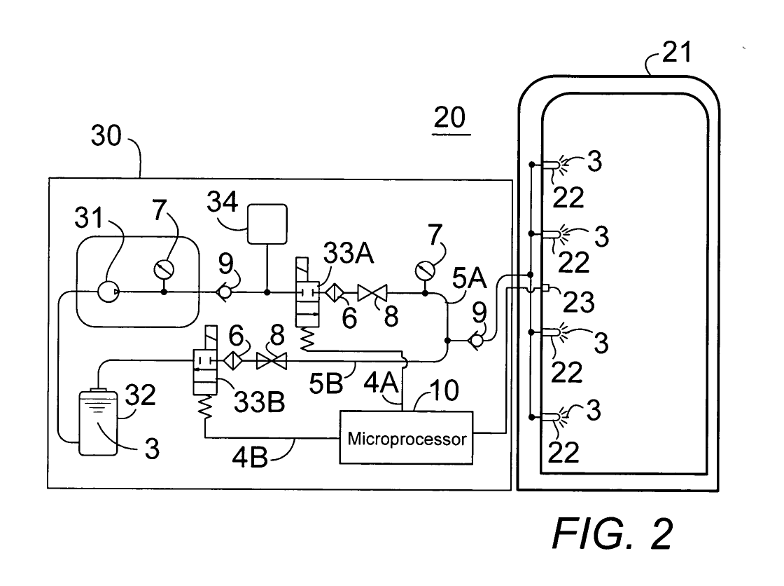

In FIGS. 1 and 2, a spray booth system 20 for cosmetic solution to simulate tanning comprises a spray booth 21 with a vertical array of stationary spray nozzles 22, a cosmetic solution tank 32A connected by a hose loop 5A and 5B to the nozzles and back into the cosmetic solution tank 32A, a water tank 32B connected through a pressurized hose 5C to a water nozzle 22A mounted on a ceiling inside the spray booth, a drain opening 24 in a floor of the spray booth connected by a pressurized line to a drainage tank 32C, and a microprocessor 10 controlling solenoid valves 33A-33D to control the flow of the liquids through the system.

The spray booth 21 comprises an enclosure adapted to receive a user standing in the booth, as seen in FIG. 1, and adapted to retain liquid within the spray booth and at least one stationary spray nozzle 22, but preferably a vertical array of stationary nozzles 22 spaced apart on at least one wall of the booth, the vertical array of nozzles adapted for providing a combined quantity of spray of the cosmetic solution sufficient to cover an entire length of a body of a user to simulate a tan on a body of a user.

In FIG. 2, at least one cosmetic solution tank 32A is positioned exteriorly of the spray booth, preferably in an enclosure 30. The cosmetic solution tank 32A is adapted for containing a cosmetic solution 3 to simulate tanning. At least one solution transmission line connects the cosmetic solution tank 32A and the array of nozzles 22 in the spray booth 21, with the line preferably formed in a loop with a first hose 5A into the spray booth 21 and a second hose 5B back to the cosmetic solution tank 32A together forming a loop adapted for transmitting the cosmetic solution therebetween.

At least one pressure tank 34 positioned exteriorly of the spray booth 21 communicates with the solution transmission line 5A and 5B and pressurizes the line to cause the cosmetic solution to flow therethrough. At least one electric pump 31A positioned exteriorly of the spray booth 21 pressurizes the pressure tank 34.

A solenoid control valve 33A, positioned in the cosmetic solution transmission line 5A leading into the spray booth 21, is adapted for opening to admit a flow of the cosmetic solution through the cosmetic solution transmission line 5A and closing to prevent a flow of the cosmetic solution through the solution transmission line. A second solenoid control valve 33B in the cosmetic solution transmission line 5B back to the cosmetic solution tank 32A is adapted for admitting a flow of the cosmetic solution from the nozzles and the first half of the loop 5A to the cosmetic solution tank 32A after the flow into the booth stops.

A microprocessor 10 is interconnected by electric signal transmission lines 4A-4D to the solenoid valves 33A-33D. The microprocessor is programmable to control the solenoid valves to control the frequency and duration of the flow of the cosmetic solution to provide a desired amount of the cosmetic solution to the user;

An activation control 23 is positioned within the spray booth accessible to a user in the booth. The activation control is interconnected with the microprocessor 10 for activating the microprocessor to activate the system for providing the cosmetic solution to the user through the array of nozzles 22 as a quiet automated spray.

The system further comprises at least one water tank 32B positioned exteriorly of the spray booth and the spray booth 21 further comprises at least one water spray nozzle 22A mounted in an elevated position, preferably on the ceiling or high on the wall in the spray booth 21, the spray nozzle adapted for spraying a quantity of water into the spray booth to wash the booth clean.

A water transmission line 5D interconnects the water tank 32B and at least one water spray nozzle 22A in the spray booth for transmitting a quantity of water therebetween. An electric pump 31C is adapted for pressurizing the water tank 32B. A solenoid control valve 33C positioned in the water transmission line 5C is adapted for opening to admit a flow of a quantity of water 13 through the water transmission line 5D.

The system 20 further comprises at least one drainage tank 32C interconnected by a drainage transmission line 5D to a drain opening 24 mounted in a low position in the spray booth, preferably set into a sloping floor, the drainage transmission line adapted for transmitting a quantity of drainage water therebetween. An electric pump 31C is adapted for pressurizing the at least one drainage water tank 32C to draw waste water 16 out of the spray booth 21 through the drainage opening 24.

Various check valves 9 and pressure gauges 7 and filters 6 are used to control each of the flows more effectively and keep the cosmetic solution filtered. Manual shutoffs 8 are also provided to override the programmed system when necessary.

In use, a user enters the spray booth 21 wearing goggles and a hair cover. Pressing the activation control 23 activates the microprocessor 10 to open the solenoid control valve 33A to admit a flow of cosmetic solution 3 to spray through the nozzles 22 for a programmed amount of time to cover one side of the user from head to toe. At the expiration of the time, the microprocessor is programmed to close the first solenoid control valve 33A and open the second solenoid control valve 33B in the cosmetic solution loop to allow the cosmetic solution 3 in the loop, including hoses 5A and 5B, to flow back into the cosmetic solution tank 32A.

The user then turns around and the process is repeated for the rest of the body of the user. After a programmed period of time after the completion of the second cosmetic solution spray cycle, providing sufficient time for the user to exit the spray booth, the water solenoid control valve 33C is opened to force water 13 under pressure through the water transmission line 5C and through the water spray valve 22A to clean out the spray booth for the next user. A fourth solenoid control valve 33D in the drainage line 5D is opened to drain the spray booth of waste water 16 through the drain opening 24 in the bottom of the spray booth and into the drainage tank 32C.

It is understood that the preceding description is given merely by way of illustration and not in limitation of the invention and that various modifications may be made thereto without departing from the spirit of the invention as claimed.

1. A spray booth system for cosmetic solution to simulate tanning, the system comprising:
a spray booth comprising an enclosure adapted to receive a user standing in the booth and adapted to retain liquid within the booth and at least one stationary spray nozzle mounted in a stationary position in the booth, the at least one nozzle adapted for spraying a body of a user with a cosmetic solution to simulate a tan on a body of a user;
at least one cosmetic solution tank positioned exteriorly of the booth, the at least one cosmetic solution tank adapted for containing a cosmetic solution to simulate tanning and at least one solution transmission line interconnected between the at least one tank and the at least one nozzle in the booth, the at least one solution transmission line adapted for transmitting the cosmetic solution therebetween;
at least one pressure tank positioned exteriorly of the booth, the at least one pressure tank communicating with the at least one solution transmission line, the at least one pressure tank adapted for pressurizing the at least one solution transmission line to cause the cosmetic solution to flow therethrough;
at least one electric pump positioned exteriorly of the booth, the at least one electric pump adapted for pressurizing the at least one pressure tank;
at least one solenoid control valve positioned in the at least one solution transmission line, the at least one solenoid control valve adapted for opening to admit a flow of the cosmetic solution through the at least one solution transmission line and closing to prevent a flow of the cosmetic solution through the at least one solution transmission line;
a microprocessor interconnected by an electric signal transmission line with the at least one solenoid, the microprocessor being programmable to control the at least one solenoid to control the frequency and duration of the flow of the cosmetic solution to provide a desired amount of the cosmetic solution to the user;
an activation control positioned within the booth accessible to a user in the booth, the activation control interconnected with the microprocessor for activating the microprocessor to activate the system for providing the cosmetic solution to the user through the at least one nozzle as a quiet automated spray controlled by the microprocessor.

2. The system of claim 1 wherein the at least one nozzle comprises a vertical array of stationary nozzles spaced apart on at least one wall of the booth, the vertical array of nozzles adapted for providing a combined quantity of spray of the cosmetic solution sufficient to cover an entire length of a body of a user.

3. The system of claim 1 wherein the at least one solution transmission line comprises an input solution hose with a first solenoid control valve adapted for admitting flow of the cosmetic solution from the at least one cosmetic solution tank to the at least one nozzle and an output solution hose with a second solenoid control valve adapted for admitting a flow of the cosmetic solution from the at least one nozzle to the at least one cosmetic solution tank, the two hoses interconnected in a loop, so that the microprocessor is programmable to open the first solenoid control valve to allow a programmed spray of the cosmetic solution sufficient to cover a length of a body of a user and programmable to close the first solenoid control valve and open the second solenoid control valve to allow the cosmetic solution in the loop to flow back into the at least one cosmetic solution tank.

4. The system of claim 1 further comprising:
at least one water tank positioned exteriorly of the spray booth and the spray booth further comprising at least one water spray nozzle mounted in an elevated position in the spray booth, the at least one water spray nozzle adapted for spraying a quantity of water into the spray booth to wash the booth clean;
at least one water transmission line interconnected between the at least one tank and the at least one water spray nozzle in the booth, the at least one solution transmission line adapted for transmitting a quantity of water therebetween;
at least one electric pump positioned exteriorly of the booth, the at least one electric pump adapted for pressurizing the at least one water tank;
at least one solenoid control valve positioned in the at least one water transmission line, the at least one solenoid control valve adapted for opening to admit a flow of a quantity of water through the at least one water transmission line.

5. The system of claim 1 further comprising:
at least one drainage tank positioned exteriorly of the spray booth and the spray booth further comprising at least one drain opening mounted in a low position in the spray booth;
at least one drainage transmission line interconnected between the at least one tank and the at least one drain opening in the booth, the at least one drainage transmission line adapted for transmitting a quantity of drainage water therebetween;
at least one electric pump positioned exteriorly of the booth, the at least one electric pump adapted for pressurizing the at least one drainage water tank;
at least one solenoid control valve positioned in the at least one drainage transmission line, the at least one solenoid control valve adapted for opening to admit a flow of a quantity of drainage water through the at least one drainage transmission line from the drain opening into the drainage tank.
